(19) United States
(12) Patent Application Publication
(10) Pub. No.: US 2014/0234227 A1
(43) Pub. Date: Aug. 21, 2014

(54) FOAM FORMULATIONS

(71) Applicant: OTIC PHARMA LTD, Rehovot (IL)

(72) Inventors: Rodrigo Yelin, Kfar Yona (IL); Jorge Danziger, Rishon Le Zion (IL); Eran Eilat, Herzliya (IL)

(73) Assignee: OTIC PHARMA LTD., Rehovot (IL)

(21) Appl. No.: 14/350,948

(22) PCT Filed: Oct. 10, 2012
(86) PCT No.: PCT/IL2012/050401

§ 371 Date: Apr. 10, 2014

Related U.S. Application Data
(60) Provisional application No. 61/545,209, filed on Oct. 10, 2011.

(51) Int. Cl. A61K 31/573; A61K 9/12; A61K 31/496

(52) U.S. Cl. CPC A61K 31/573; A61K 31/496; A61K 9/122
USPC 424/43

(57) ABSTRACT

The present invention provides foamable pharmaceutical compositions comprising water-soluble antibiotics and anti-inflammatory steroids and methods of preparing same. Particularly, the present invention relates to oil-free pharmaceutical compositions comprising a quinolone, an anti-inflammatory steroid, a polar co-solvent, and propellant gas, administered to the ear in the form of foam for treating ear disorders.

### FIELD OF THE INVENTION

The present invention relates to foamable pharmaceutical compositions comprising water-soluble antibiotics together with anti-inflammatory steroids and methods of preparing same. Particularly, the present invention relates to oil-free pharmaceutical compositions comprising quinolones, anti-inflammatory steroids, polar co-solvents, water, and propellant gas, suitable for administration to the ear in the form of foam for treating ear disorders.

### BACKGROUND OF THE INVENTION

Otitis externa which involves the ear canal of the external ear is a common otologic problem occurring mainly during hot and humid weather. Otitis externa is five times more frequent in swimmers than in non-swimmers. It is an acute or chronic inflammation of the epithelium of the external ear canal. It may develop anywhere from the tympanic membrane to the pinna. It is variably characterized by erythema, edema, increased sebum or exudates, and desquamation of the epithelium. In later stages, suppuration occurs in the ear canal and hearing may be decreased. Over 90% of cases of acute Otitis Externa (AOE) are due to bacterial infections and more rarely to fungal infections.

Otitis media involves infections of the middle ear and it is a very common otologic problem in adults and particularly in children. It has been estimated that nearly 95% of all children experience one or more episodes of otitis by age 9, and that about 15% of all visits by children to pediatricians are in regard to otitis media. In children, the disease is often associated with upper respiratory tract infections which trigger a transudate secretion response in the Eustachian tube and middle ear. Bacteria and viruses migrate from the naso-pharynx to the middle ear via the Eustachian tube, and can cause the Eustachian tube to become blocked, preventing ventilation and drainage of the middle ear.

Otitis externa is the most common disease of the ear canal in dogs and cats, and is occasionally seen in rabbits (in which it is usually due to the mite Psoroptescuniculi).

The common treatment of AOE consists of topical antibiotics, with or without steroids, analgesia and water avoidance. Otic preparations are generally supplied in the form of ear drops. In advanced cases, when suppuration blocks the external ear canal, a wick is inserted and soaked in the topical preparation. However, its insertion may be traumatic, inconvenient and painful. Antibiotic agents commonly used in ear drops include aminoglycosides (mainly neomycin) in combination with polymyxin B and hydrocortisone or fluoroquinolones such as ciprofloxacin and ofloxacin. The incidence of hypersensitivity reactions to neomycin-containing ear drops has been reported to be as high as 13%, while such reactions to fluoroquinolones have been reported to be rare. In addition, aminoglycosides have the potential to damage the inner ear when the tympanic membrane is perforated, while ototoxicity is not a concern with fluoroquinolones.

Ciprofloxacin is a safe and efficacious antibacterial fluoroquinolone active against a broad spectrum of gram-positive and gram-negative bacteria. Ciprofloxacin is present as Ciprofloxacin base and Ciprofloxacin HCl. Ciprofloxacin HCl is the monohydrochloride monohydrate salt of 1-cyclopropyl-6-fluoro-1,4-dihydro-4-oxo-7-(1-piperazinyl)-3-quinolinecarboxylic acid with the following chemical structure:

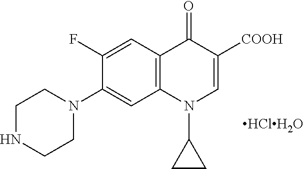 [SMILES: O=C(O)c1cn(C2CC2)c2cc(N3CCNCC3)c(F)cc2c1=O]

Due to its proven safety and lack of ototoxicity, ciprofloxacin ear drops are prescribed to treat patients with Acute Otitis Externa with intact or non-intact tympanic membrane. For example, topical otic compositions containing a combination of either ciprofloxacin and hydrocortisone or ciprofloxacin and dexamethasone are sold in the forms of aqueous solutions or suspensions.

U.S. Pat. No. 5,061,729 to Kincses et al. discloses ear drop compositions for the treatment of chronic otitis media which consists essentially of: (a) 3 to 19% of a sulfhydryl compound selected from the group consisting of N-acetyl-L-cysteine, disulfiram, and pantetheine; (b) an antibacterial agent selected from the group consisting of tobramycin, neomycin, polymixin B, gentamycin, a combination of tobramycin and neomycin in a 1:1 weight ratio, and a combination of polymixin B and neomycin, tobramycin, or gentamycin in a ratio of 100,000 IU to about 100 mg; (c) an anti-inflammatory agent selected from the group consisting of hydrocortisone, mazipredone, beclomethasone dipropionate, triamcinolone acetonide, prednisolone, dexamethasone, and betamethasone; and (d) the balance of an aqueous solution of propylene glycol; in admixture with zinc oxide in an amount effective to stabilize the sulfhydryl compound.

U.S. Pat. No. 5,843,930 to Purwar et al. discloses a non-ototoxic, topical, otic pharmaceutical composition for the treatment of otitis which comprises ciprofloxacin in aqueous solution, a non-ionic viscosity augmenter being at least 85% hydrolyzed polyvinyl alcohol, a non-ototoxic preservative, water sufficient to produce an aqueous composition, hydrocortisone in aqueous suspension, lecithin in an amount effective for enhancing suspension of other constituents, and polysorbate 20 to 80 in an amount effective for spreading the composition on a hydrophobic skin surface. U.S. Pat. No. 5,843,930 further discloses methods for treating otitis which comprise administering the otic pharmaceutical composition topically into the ear.

U.S. Pat. No. 6,284,804 to Singh et al. discloses suspension formulations comprising dexamethasone, ciprofloxacin, sodium chloride as an ionic tonicity agent, a nonionic polymer, and a nonionic surfactant. According to U.S. Pat. No. 6,284,804, dexamethasone can be present in any ophthalmically or otically acceptable form having poor water solubility such that the resulting formulation is a suspension formulation.

U.S. Pat. No. 6,462,033 to Singh discloses a method of preparing a topical composition comprising ciprofloxacin and hydrocortisone. The method according to U.S. Pat. No. 6,462,033 involves dispersing hydrocortisone with lecithin for greater than 45 minutes prior to combining hydrocortisone with the remainder of the composition.

U.S. Pat. No. 6,919,069 to Akehurst et al. discloses aerosol formulations comprising particulate steroid drugs, a polar co-solvent, and a propellant. U.S. Pat. No. 6,919,069 further discloses methods for treating respiratory disorders comprising administering said formulations by inhalation.

U.S. Pat. No. 7,704,518 to Tamarkin et al. discloses a hygroscopic pharmaceutical composition which includes at least one hygroscopic substance at a concentration sufficient to provide an Aw value of at least 0.9 and an anti-infective agent. U.S. Pat. No. 7,704,518 further discloses a foamable pharmaceutical carrier which includes about 50% to about 98% of a polar solvent selected from the group consisting of a polyol and a polyethylene glycol; 0% to about 48% of a secondary polar solvent; about 0.2% to about 5% by weight of a surface-active agent; about 0.01% to about 5% by weight of at least one polymeric agent; and a liquefied or compressed gas propellant at a concentration of about 3% to about 25% by weight of the total composition. According to U.S. Pat. No. 7,704,518, the compositions further comprise up to 10% water.

U.S. Pat. No. 7,973,022 to Murthy discloses pharmaceutical compositions that are a solution of a salt formed between a fluoroquinolone and a carboxylic acid, a cyclodextrin, and a pharmaceutically acceptable organic carrier. The pharmaceutical compositions according to U.S. Pat. No. 7,973,022 are substantially free of water and are useful for treating a condition in an animal comprising orally administering or injecting the composition to the animal.

U.S. Pat. No. 8,003,616 to Albert et al. discloses a composition for treating ear infections in animals which includes an antifungal agent, an antibiotic agent, a steroidal anti-inflammatory agent and an acid dissolved to form a liquid. The method for preparing said composition includes dissolving the antifungal agent, steroidal anti-inflammatory agent, and acid in dehydrated alcohol, dissolving the antibiotic agent in propylene glycol, combining the two solutions together, heating the combination and then cooling the composition. According to U.S. Pat. No. 8,003,616, it is best to prepare the composition in a low-humidity environment to avoid the introduction of water into the composition during formulation.

U.S. Patent Application Publication No. 2006/0269485 to Friedman et al., discloses a foamable composition which includes: an antibiotic agent; at least one organic carrier selected from the group consisting of a hydrophobic organic carrier, an organic polar solvent, an emollient, and mixtures thereof, at a concentration of about 2% to 50% by weight; a surface active agent at a concentration of about 0.1% to about 5% by weight; at least one polymeric additive selected from the group consisting of a bioadhesive agent, a gelling agent, a film forming agent, and a phase change agent; water; and liquefied or compressed gas propellant at a concentration of about 3% to about 25% by weight of the total composition. According to U.S. Patent Application Publication No. 2006/029485, the composition is an oil-in-water emulsion or water-in-oil emulsion.

International Application Publication No. WO 2005/055921 to one of the inventors of the present invention discloses a pharmaceutical composition for the treatment of an ear disorder in a form selected from foam and mousse comprising: a pharmaceutical agent known to affect an ear disorder; and a pharmaceutically acceptable carrier comprising a dispersing agent that is a foam forming agent; wherein the pharmaceutical composition being dispensed from a dispensing device to the external auditory meatus.

International Application Publication No. WO 2010/143186 to the inventors of the present invention discloses pharmaceutical compositions comprising oil-in-water emulsions comprising fluoroquinolones and gas propellants, administered to the ear in the form of foam for treating ear disorders.

There is an unmet need for foamable, clinically efficacious, and stable compositions comprising a quinolone and an anti-inflammatory steroid which can be readily and compliantly used by a patient for the treatment of ear disorders.

### SUMMARY OF THE INVENTION

The present invention provides foamable antibiotic compositions which comprise a quinolone and a steroidal anti-inflammatory agent. Advantageously, the present invention provides oil-free foamable compositions comprising as active agents a quinolone and a steroidal anti-inflammatory agent, a polar co-solvent, water and a compressed gas for treating otitis. The present invention further provides methods for preparing the oil-free foamable compositions.

The present invention is based in part on the surprising finding that a foamable pharmaceutical composition which comprises a polar co-solvent and water as the major solvents/solutes in combination with surfactants, which composition is devoid of oil and further comprises a quinolone and an anti-inflammatory steroid as active agents, enables maintaining the quinolone and the anti-inflammatory steroid in a soluble form.

The present invention discloses that oil-free pharmaceutical compositions which comprise a polar co-solvent and water, wherein the polar co-solvent is present in an amount of about 40% to about 70% of the total weight of the composition and water is present in amount of about 30% to about 60% of the total weight of the composition, such compositions maintain the stability of a quinolone and a steroidal anti-inflammatory agent for at least twelve months at room temperature.

It is now disclosed that dexamethasone when dissolved in propylene glycol and mixed with ciprofloxacin hydrochloride, water, a surfactant, a foaming agent, a polymeric agent, and a compressed gas produced a foamable homogenous composition in which dexamethasone is neither aggregated/precipitated nor suspended.

The present invention further discloses that upon dispensing the foamable composition from a dispensing device into the ear canal of a subject, the composition formed a stable foam in the ear canal which collapsed within half an hour to an hour, thus providing a slow-release effect of the active agents, leading to an extended exposure of the ear canal to the active agents as compared to the effect obtained by commercially available ear drops.

Due to the fact that the foamable pharmaceutical composition of the present invention is oil-free, it advantageously does not leave oily residues at the application site.

It is further disclosed that the foamable pharmaceutical composition of the present invention provides a comfortable feeling for the user as it is devoid of short-chain alcohols. The foamable composition did not cause skin irritation or any allergic response when tested in humans and as such it is safe and effective in human or veterinary subjects.

According to one aspect, the present invention provides a foamable pharmaceutical composition comprising:
(i) a polar co-solvent in an amount of from about 40% (w/w) to about 70% (w/w) of the total weight of the composition;
(ii) water in an amount of from about 30% (w/w) to about 60% (w/w) of the total weight of the composition;
(iii) a surfactant and/or a foaming agent;
(iv) a stabilizing agent;
(v) a quinolone;
(vi) a steroidal anti-inflammatory agent; and
(vii) a propellant gas;

wherein the composition is devoid of oil, and wherein the composition packaged in a container is adapted to form foam after dispensing from the container.

It is to be understood that the foamable pharmaceutical composition of the present invention is stored in an aerosol or pressurized container. Upon dispensing from the aerosol container, the foamable pharmaceutical composition forms foam suitable for application into the ear. Thus, the foamable pharmaceutical composition of the present invention when administered into the ear of a subject, e.g., a human or an animal, is in the form of foam.

According to some embodiments, the polar co-solvent is selected from the group consisting of propylene glycol, polyethylene glycol, polypropylene glycol alkyl ether, and combinations thereof. According to a certain embodiment, the polar co-solvent is propylene glycol. According to additional embodiments, propylene glycol is present in the composition in an amount of from about 42% (w/w) of the composition to about 60% (w/w) of the composition, alternatively from about 45% (w/w) to about 52% (w/w) of the composition, and further alternatively from about 48% (w/w) to about 50% (w/w) of the composition. Each possibility represents a separate embodiment of the invention. According to an exemplary embodiment, propylene glycol is present in the composition in an amount of about 49% (w/w) of the composition.

According to further embodiments, water is present in the composition in an amount of from about 40% (w/w) to about 50% (w/w), or alternatively in an amount of from 42% (w/w) to about 48% (w/w) of the composition. Each possibility represents a separate embodiment of the invention. According to an exemplary embodiment, water is present in the composition in an amount of about 46% (w/w) of the composition.

According to additional embodiments, the polar co-solvent and water are present in the composition in a ratio of from about 2:1 to about 2:3. According to an exemplary embodiment, the polar co-solvent is propylene glycol present in a ratio of 1:1 with water.

According to further embodiments, the surfactant is a synthetic surfactant selected from the group consisting of polysorbate 20, polysorbate 60, polysorbate 80, polyoxyl 40 stearate, glyceryl stearate, and combinations thereof. Each possibility represents a separate embodiment of the invention. According to yet further embodiments, the amount of the surfactant in the pharmaceutical composition ranges from about 2% (w/w) to about 4% (w/w) of the composition. According to an exemplary embodiment, the surfactant is a mixture of polysorbate 80 and polyoxyl 40 stearate.

According to additional embodiments, the foaming agent is a long chain alcohol having at least 12 carbon atoms in the carbon chain such as cetyl alcohol and cetostearyl alcohol, and combinations thereof. According to further embodiments, the amount of the foaming agent in the pharmaceutical composition ranges from about 0.1% to about 0.5% (w/w) of the composition. According to an exemplary embodiment, the foaming agent is cetyl alcohol.

According to yet further embodiments, the stabilizing agent is a polymeric agent. Examples of polymeric agents include semi-synthetic polymers such as cellulose ethers, e.g., hydroxyethyl cellulose, methyl cellulose, carboxymethyl cellulose, and hydroxy propylmethyl cellulose; synthetic polymeric materials such as polyvinyl alcohol; and combinations thereof. Each possibility represents a separate embodiment of the 5 invention. According to still further embodiments, the amount of the stabilizing agent ranges from about 0.1% to about 0.5% (w/w) of the total weight of the composition. According to an exemplary embodiment, the stabilizing agent is hydroxyethyl cellulose.

According to some embodiments, the quinolone is selected from the group consisting of ciprofloxacin, ofloxacin, moxifloxacin, finafloxacin, levofloxacin, lomefloxacin, nadifloxacin, norfloxacin, pefloxacin, rufloxacin, balofloxacin, gatifloxacin, grepafloxacin, levofloxacin, pazufloxacin, sparfloxacin, temafloxacin, tosufloxacin, clinafloxacin, gemifloxacin, sitafloxacin, trovafloxacin, prulifloxacin, garenoxacin, delafloxacin, marbofloxacin, enrofloxacin, danofloxacin, difloxacin, ibafloxacin, orbifloxacin, sarafloxacin, and pharmaceutically acceptable salts thereof. Each possibility represents a separate embodiment of the invention. The quinolone is present in the composition in an amount effective for antibiotic action. Such amount typically ranges from about 0.1% to about 3% (w/w) of the total weight of the composition. According to a certain embodiment, the quinolone is ciprofloxacin or a salt thereof. According to an exemplary embodiment, ciprofloxacin is ciprofloxacin hydrochloride present in the pharmaceutical composition in an amount of about 0.35% (w/w) of the total weight of the composition.

According to a further embodiment, the composition is for animal use and the quinolone is marbofloxacin, orbifloxacin or enrofloxacin.

According to additional embodiments, the steroidal anti-inflammatory agent is selected from the group consisting of dexamethasone, hydrocortisone, prednisolone, methylprednisolone, prednisone, triamcinolone, mometasone, budesonide, fluocinolone, betamethasone, cortisone, isoflupredone, tixocortol, triamcinolone alcohol, amcinonide, desonide, fluocinonide, halcinonide, fluocortolone, aclometasone, clobetasone, fluocortolone, fluprednidene, and pharmaceutically acceptable salts thereof. Each possibility represents a separate embodiment of the invention. According to a certain embodiment, the anti-inflammatory steroid is dexamethasone. The anti-inflammatory steroid is present in the composition in an amount effective for anti-inflammatory action. Such amount typically ranges from about 0.05% to about 3% (w/w) of the total weight of the composition.

According to a further embodiment, the steroidal anti-inflammatory agent is present in the composition in a soluble form. Thus, the present invention provides a foamable pharmaceutical composition wherein both the quinolone and the steroidal anti-inflammatory agent are soluble in the composition.

According to yet further embodiments, the propellant gas is selected from volatile hydrocarbons such as butane, propane, isobutane, and mixture thereof. The amount of the compressed propellant gas in the pharmaceutical composition ranges from about 1% to about 8% by weight of the composition, alternatively from about 2% to about 6%, and further alternatively from about 4% to about 6% by weight of the composition. It is to be understood that gas propellants such as, for example, hydrofluoroalkanes, chlorofluoroalkanes, dimethyl ethers, and methyl ethers can be used as propellant gas in the compositions of the present invention. Each possibility represents a separate embodiment of the invention.

According to yet further embodiments, the composition can further comprise a buffering system selected from the group consisting of acetate buffer, citrate buffer, and phosphate buffer. Each possibility represents a separate embodiment of the invention. According to a certain embodiment, the buffering system comprises sodium acetate and acetic acid. According to an exemplary embodiment, the amount of sodium acetate in the pharmaceutical composition ranges from about 0.1% to about 1% (w/w) of the composition, preferably the amount is of about 0.1% to about 0.2% (w/w) of the composition.

According to still further embodiments, the pH of the pharmaceutical composition ranges from about 4 to about 7. Alternatively, the pH of the composition ranges from about 4 to about 5. According to a certain embodiment, the pH of the pharmaceutical composition ranges from about 4.5 to about 5. Each possibility represents a separate embodiment of the invention.

According to additional embodiments, the foamable pharmaceutical composition can further comprise a preservative.

According to yet further embodiments, the foamable pharmaceutical composition can further comprise an analgesic agent. According to still further embodiments, the composition is for animal use and can further comprise an antifungal agent.

According to yet further embodiments, the foamable pharmaceutical composition is for animal use and can further comprise an insecticide agent.

According to certain embodiments, the foamable pharmaceutical composition comprises:
(i) propylene glycol as a polar co-solvent present in an amount of from about 40% (w/w) to about 70% (w/w) of the total weight of the composition;
(ii) water present in an amount of from about 30% (w/w) to about 60% (w/w) of the total weight of the composition;
(iii) a surfactant and/or a foaming agent;
(iv) a stabilizing agent;
(v) ciprofloxacin;
(vi) dexamethasone base; and
(vii) propellant gas;

wherein the composition is devoid of oil, and wherein the composition packaged in a container is adapted to form foam after dispensing from the container.

According to additional embodiments, the foamable pharmaceutical composition optionally further comprises a second polar co-solvent.

According to an exemplary embodiment, the foamable pharmaceutical composition comprises:

| Ingredient | w/w (%) |
|---|---|
| Propylene glycol | 49.00 |
| Water | 46.38 |
| Polysorbate 80 | 2.50 |
| PEG 40 stearate | 0.80 |
| Cetyl alcohol | 0.40 |
| Hydroxyethyl cellulose | 0.20 |
| Glycerol | 0.10 |
| Ciprofloxacin HCl | 0.35 |
| Dexamethasone base | 0.10 |
| Sodium acetate | 0.15 |
| Acetic acid | q.s. |
| Benzalkonium chloride | 0.02 |

q.s. = quantity sufficient to obtain the desired pH.

According to another aspect, the present invention provides a pharmaceutical composition comprising:
(i) a polar co-solvent in an amount of from about 40% (w/w) to about 70% (w/w) of the total weight of the composition;
(ii) water in an amount of from about 30% (w/w) to about 60% (w/w) of the total weight of the composition;
(iii) a surfactant and/or a foaming agent;
(iv) a stabilizing agent;
(v) a quinolone;
(vi) a steroidal anti-inflammatory agent

According to a further aspect, the present invention provides a method for treating an ear disorder comprising administering to the ear of a subject in need of such treatment a therapeutically effective amount of the foamable pharmaceutical composition according to the principles of the present invention. According to one embodiment, the subject is a human. According to another embodiment, the subject is an animal. According to a further embodiment, the animal is a domestic pet animal. According to certain embodiments, the domestic pet animal is a dog or a cat.

According to additional embodiments, the ear disorder is otitis selected from the group consisting of otitis externa and otitis media. According to further embodiments, the foamable pharmaceutical composition is administered to the ear canal and the otitis externa is selected from the group consisting of acute otitis externa and suppurative otitis externa. According to further embodiments, the otitis media is selected from the group consisting of chronic suppurative otitis media and otitis media in the presence of tympanostomy tubes.

According to yet further aspect, the present invention provides a foamable pharmaceutical composition according to the principles of the present invention for the treatment of an ear disorder.

These and other embodiments of the present invention will be better understood in relation to the description, examples and claims that follow.

### DETAILED DESCRIPTION OF THE INVENTION

The present invention provides a foamable pharmaceutical composition comprising: (i) a polar co-solvent in an amount of from 40% (w/w) to 70% (w/w) of the total weight of the composition; (ii) water in an amount of from 30% (w/w) to about 60% (w/w) of the total weight of the composition; (iii) a surfactant and/or a foaming agent; (iv) stabilizing agent; (v) a quinolone; (vi) a steroidal anti-inflammatory agent; and (vii) a compressed propellant gas; wherein the composition is devoid of oil, and wherein the composition packaged in a container is adapted to form foam after dispensing from the container.

The compositions of the present invention are devoid of an oil component and as such do not form oil-in-water emulsions or water-in-oil emulsions as are known in the art. As used herein the term “oil” refers to liquid oil that may originate from vegetable, marine or animal sources. Alternatively, the oil can be a mineral oil. Mineral oil is a mixture of aliphatic, naphthalenic, and aromatic liquid hydrocarbons that derive from petroleum. The term “oil” further refers to polyunsaturated oils containing poly-unsaturated fatty acids, and to triglycerides, i.e., triesters of glycerin and fatty acids.

Advantageously, the steroidal anti-inflammatory agent and the quinolone are present in a soluble form in the compositions of the present invention, thus providing long stability and accordingly long shelf-life of the compositions.

The term “soluble” refers herein to a compound having solubility of at least 1 gr/30 ml of the composition at room temperature. A quinolone is preferably water-soluble, i.e., having solubility in water of at least 1 gr/30 ml at room temperature. It is to be understood that according to the principles of the present invention, the steroidal anti-inflammatory agent is preferably soluble in the organic co-solvents such as propylene glycol and polyethylene glycol, e.g., PEG-400. Accordingly, both the quinolone and the anti-inflammatory agent are preferably soluble in the composition. According to an exemplary embodiment, the pharmaceutical composition is formulated as a solution.

Thus, the present invention further provides a foamable pharmaceutical composition comprising: (a) a solution comprising: (i) a polar co-solvent in an amount of from 40% (w/w) to 70% (w/w) of the total weight of the composition; (ii) water in an amount of from 30% (w/w) to about 60% (w/w) of the total weight of the composition; (iii) a surfactant and/or a foaming agent; (iv) stabilizing agent; (v) a quinolone; (vii) a steroidal anti-inflammatory agent; and (b) a compressed propellant gas; wherein the composition is devoid of oil, and wherein the composition packaged in a container is adapted to form foam after dispensing from the container.

The present invention further provides foamable pharmaceutical compositions for use in treating ear disorders in humans and in animals. It is to be understood that the compositions for animal use can further comprise additional agents selected from anti-fungal agents, insecticides and/or miticide agents.

The term “about” as used herein denotes ±10% of the value indicated.

The term “therapeutically effective amount” is that amount of the active agent which is sufficient to provide a beneficial effect to the subject to which the active agent is administered. More specifically, a therapeutically effective amount means an amount of a quinolone effective to alleviate or ameliorate the symptoms of an ear disorder of the subject being treated. Among the symptoms of an ear disorder, ear edema, ear pain, ear discharge, and tenderness to movement of the tragus/pinna are more typical.

The term “pharmaceutically acceptable salt” refers to salts prepared from pharmaceutically acceptable non-toxic bases or acids including inorganic or organic bases and inorganic or organic acids.

The composition comprises a polar co-solvent. A “polar co-solvent” is an organic solvent, typically soluble in both water and oil. Examples of polar co-solvents include, but are not limited to, polyols, such as glycerol (glycerin), propylene glycol (PPG), hexylene glycol, diethylene glycol, PPG n-alkanols, and (PPG) alkyl ether. According to a certain embodiment, the polar co-solvent is a polyethylene glycol (PEG), PPG, or a derivative thereof that is liquid at ambient temperature, including, but not limited to, PEG200 (molecular weight (MW) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

Polar co-solvents also include propylene glycol (e.g., 1,2-propylene glycol and 1,3-propylene glycol), butanediol (e.g., 1,2-butanediol, 1,3-butanediol, 2,3-butanediol and 1,4-butanediol), butynediol, pentanediol (e.g., pentane-1,2-diol, pentane-1,3-diol, pentane-1,4-diol, pentane-1,5-diol, pentane-2,3-diol and pentane-2,4-diol), hexanediol (e.g., hexane-1,6-diol hexane-2,3-diol and hexane-2,56-diol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

Additional examples of polar co-solvents include butane-1,2,3-triol, butane-1,2,4-triol and hexane-1,2,6-triol. It is to be understood that the compositions of the present invention can comprises a mixture of polar co-solvents.

The stabilizing agent is a polymeric agent that increases the viscosity of the composition, contributes to the composition stability, and/or slows the foam collapse rate. Examples of stabilizing agents include, but are not limited to, naturally-occurring polymeric materials (e.g., alginate, albumin, gelatin, carrageenan, xanthan gum, starch), semi-synthetic polymeric materials such as cellulose ethers (e.g. hydroxyethyl cellulose, methyl cellulose, carboxymethyl cellulose, hydroxy propylmethyl cellulose), and synthetic polymeric materials (e.g. polyvinyl alcohol, carboxyvinyl polymers, and polyvinylpyrrolidone).

A surfactant or a surface-active agent include any agent suitable for linking two liquids. Non-limiting examples of possible surfactants are synthetic non-ionic surfactants that include polysorbates, such as polyoxyethylene sorbitan monolaurate (Tween 20), polyoxyethylene sorbitan monostearate (Tween 60) and polyoxyethylene sorbitan monooleate (Tween 80); glyceryl stearate; polyoxyethylene (POE) fatty acid esters, such as Myrj 45, Myrj 49, Myrj 52 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, and the like, and a combination thereof.

The quinolone is selected from the group consisting of ciprofloxacin, ofloxacin, moxifloxacin, finafloxacin, levofloxacin, lomefloxacin, nadifloxacin, norfloxacin, pefloxacin, rufloxacin, balofloxacin, gatifloxacin, grepafloxacin, levofloxacin, pazufloxacin, sparfloxacin, temafloxacin, tosufloxacin, clinafloxacin, gemifloxacin, sitafloxacin, trovafloxacin, prulifloxacin, garenoxacin, delafloxacin, marbofloxacin, enrofloxacin, danofloxacin, difloxacin, ibafloxacin, orbifloxacin, sarafloxacin, and pharmaceutically acceptable salts thereof. For animal use, the quinolone can be marbofloxacin, orbifloxacin or enrofloxacin.

The steroidal anti-inflammatory agent is selected from the group consisting of dexamethasone, hydrocortisone, prednisolone, methylprednisolone, prednisone, triamcinolone, mometasone, budesonide, fluocinolone, betamethasone, cortisone isoflupredone tixocortol, triamcinolone alcohol, amcinonide, desonide, fluocinonide, halcinonide, fluocortolone, aclometasone, clobetasone, fluocortolone, and pharmaceutically acceptable salts thereof.

Examples of pharmaceutically acceptable salts of steroidal anti-inflammatory agents include, but are not limited to, dexamethasone acetate, hydrocortisone acetate, triamcinolone acetonide, fluocinolone acetonide, betamethasone valerate, cortisone acetate, isoflupredone acetate, tixocortol pivalate, hydrocortisone-17-butyrate, hydrocortisone-17-valerate, aclometasone dipropionate, betamethasone dipropionate, clobetasone-17-butyrate, clobetasol-17-propionate, fluocortolone caproate, fluocortolone pivalate, and fluprednidene acetate.

The analgesic agents include, but are not limited to, benzocaine, tetracaine, procaine and antipyrine.

The antifungal agents include, but are not limited to, nystatin, clotrimazole, miconazole, ketoconazole, fluconazole, thiabendazole, econazole, clomidazole, isoconazole, tiabendazole, tioconazole, sulconazole, bifonazole, oxiconazole, fenticonazole, omoconazole, sertaconazole, and flutrimazole. According to certain embodiments, nystatin, clotrimazole, miconazole, ketoconazole, fluconazole, thiabendazole are preferred.

The composition of the present invention can further comprise an emulsifier as known in the art. It is to be understood that the emulsifier in the compositions of the present invention enables formation of a clear hydrophobic or organic (oil-free) solution. The emulsifier is selected from the group consisting of lecithin or a derivative thereof, phospholipids, and fatty alcohols having 12 or more carbons in their carbon chain, such as lauryl alcohol, myristyl alcohol, cetyl alcohol, stearyl alcohol, oleyl alcohol, and linoleyl alcohol, or mixtures thereof. Other examples of fatty alcohols are arachidyl alcohol (C20), behenyl alcohol (C22), 1-triacontanol (C30), as well as alcohols with 25 longer carbon chains (up to C50). Such fatty alcohols can be useful as foaming agents as well.

The composition of the present invention can further comprise a variety of formulation excipients. Such excipients can be selected, for example, from preservatives (e.g., benzyl alcohol, benzalkonium chloride, and the like), buffering agents (acetate buffer, citrate buffer, phosphate buffer, and the like) antioxidants, colorant and odorant agents and other formulation components used in the art of formulation.

Gas propellants are used to generate and administer the foamable composition as foam. Examples of suitable gas propellants include volatile hydrocarbons such as butane, propane, isobutane or mixtures thereof, and fluorocarbon gases. The amount of the compressed propellant or liquefied gas is adapted to provide foam collapse within about 30 minutes to about 3 hours after administration of the foam into the ear canal of a subject. Alternatively, the amount of the compressed propellant or liquefied gas is adapted to provide foam collapse within about 30 minutes to about 1 hour after administration of the foam into the ear canal of a subject.

The term “foam collapse” denotes the kinetics of foam coarsening and destruction resulting from the rupture of bubbles or from gas transfer from small to large bubbles and bubble coalescence occurring within the ear of a subject.

The present invention further provides a method for preparing a foamable pharmaceutical composition, the method comprising the following steps:
(a) preparing an aqueous solution comprising water, a stabilizing agent, and optionally a polar co-solvent;
(b) preparing an organic solution comprising a surfactant and a foaming agent;
(c) combining the organic solution of step (b) with the aqueous solution of step (a) to obtain a water blend;
(d) admixing a quinolone with the water blend of step (c), optionally admixing a preservative and a buffering agent to prepare an antibiotic solution;
(e) dissolving a steroidal anti-inflammatory agent in a polar co-solvent to prepare a steroid solution;
(f) admixing the antibiotic solution of step (d) with the steroid solution of step (e) to prepare an antibiotic-steroid composition.

The foamable pharmaceutical compositions of the present invention can be useful for treating skin disorders and disorders of other body cavities such as the vagina, the rectum, the nose and the mouth. Examples of disorders that can be treated with the compositions of the present invention include, but are not limited to, skin bacterial infections, bacterial infections of the vagina such as bacterial vaginosis, anal abscess/fistula, anal fissure and anal warts.

### Example 1

### Preparation of the Formulation of Ciprofloxacin and Dexamethasone

### Aqueous Solution

Hydroxyethyl cellulose (Natrosol™ HX250) was added to water under mixing for at least 10 minutes at room temperature. Hydroxyethyl cellulose (Natrosol™ HHX250) was then added and the mixing continued until the particles were evenly dispersed (clear solution). Glycerine was added and the solution was heated to 65-70° C.

### Organic Solution

Cetyl alcohol, Polysorbate 80 and PEG 40 Stearate were added under mixing and were heated to 65-70° until clear solution was obtained.

### Addition of the Organic Solution to the Aqueous Solution

The organic solution was added to the aqueous solution under mixing at 65-70° C. for 10 minutes to form the water blend solution. The water blend solution was then cooled down to 45-50° C.

### Addition of Ciprofloxacin HCl

Benzalkonium chloride was added to the water blend solution under mixing at 40-45° C. Sodium acetate was then added under the same conditions. Water was added to complete the volume and the pH was adjusted to 4.75 (±0.1) with acetic acid. Ciprofloxacin HCl was added under mixing for at least 10 minutes at 40° C. to yield the Ciprofloxacin solution.

### Propylene Glycol Solution

Propylene glycol was heated to 40-45° C. under mixing. Dexamethasone base was added to the propylene glycol and mixed until complete dissolution.

### Addition of the Ciprofloxacin Solution to the Propylene Glycol Solution

The ciprofloxacin solution was added to the propylene glycol solution under mixing while adjusting the pH to 4.75±0.1. The solution was cooled down to room temperature under mixing and then stored at 4° C.

The formulation thus prepared included the ingredients as listed below:

| | Ingredient | w/w (%) |
|---|---|---|
| Organic | Tween 80 (surfactant) | 2.500 |
| Solution | Cetyl Alcohol (emulsifier, foaming agent) | 0.400 |
| | PEG 40 Stearate (surfactant) | 0.800 |
| Aqueous | Hydroxyethylcellulose HX (non-ionic polymer) | 0.150 |
| solution | Hydroxyethylcellulose HHX (non-ionic polymer) | 0.050 |
| | Glycerin (polar co-solvent) | 0.100 |
| | Water | 46.380 |
| | Ciprofloxacin HCl | 0.350 |
| | Benzalkonium Chloride (preservative) | 0.020 |
| | Sodium Acetate (buffering agent) | 0.150 |
| | Acetic Acid (buffering agent) | q.s. |
| Propylene | Propylene Glycol (polar co-solvent) | 49.000 |
| glycol | Dexamethasone Base | 0.100 |
| Solution | | |

q.s. = quantity sufficient to obtain the desired pH.

### Example 2

### Formulation Stability at Accelerated Aging Conditions

Stability studies at accelerated aging conditions were performed on the formulation of Example 1 designated CDX24. The formulation was packed in glass vials and in aluminum canisters coated with various varnishes, crimped with commercial valves and 10 incubated at 4° C., 25° C., 40° C. and at 50° C. for different periods of time. Storage for 1 month at 50° C. was indicative of 6 months storage at room temperature. Samples from the formulations were taken at zero time (0 months) and at the indicated time points and tested for the content of dexamethasone and ciprofloxacin by HPLC. Table 1 summarize the content of dexamethasone and ciprofloxacin in the formulations tested upon 15 incubation at accelerated and regular aging conditions.

TABLE 1
Ciprofloxacin and Dexamethasone content at different
aging conditions.

| | Zero | 1 Month | | | | 3 Months | |
|---|---|---|---|---|---|---|---|
| | Time | 4° C. | 25° C. | 40° C. | 50° C. | 4° C. | 25° C. |
| Ciprofloxacin | 94.90 | 97.00 | 97.90 | 96.96 | 98.31 | 97.53 | 99.06 |
| content | | | | | | | |
| (percentage of | | | | | | | |
| label) | | | | | | | |
| Dexamethasone | 93.90 | 98.60 | 96.72 | 93.42 | 90.77 | 98.2 | 93.36 |
| content | | | | | | | |
| (percentage of | | | | | | | |
| label) | | | | | | | |

The results presented in Table 1 show that the amount of dexamethasone did not significantly change upon incubation at 40° C. or 50° C. for 1 month. The results thus demonstrate that the formulation developed was stable and displayed a long shelf-life.

In addition, samples of the formulation were stored at 25° C. for 12 months and thereafter the content of ciprofloxacin and dexamethasone were determined by HPLC. The results indicated that the content of ciprofloxacin and dexamethasone ranged between 90% to 110% of the starting content (ciprofloxacin HCl 0.35%, dexamethasone 0.1%), indicating that the formulation was stable and the degradation of the active ingredients was within the Pharmacopeia requirements.

### Example 3

### Assessment of the Safety of the Formulation in Humans

The otic formulation designated CDX24 was tested for dermal irritation in humans. The test challenged the development of Allergic Contact Dermatitis upon application of a test item under exaggerated exposure conditions.

In a study performed in compliance with Good Clinical Practice standards, the formulation of Example 1 was applied on the interscapular region of the back or on the 20 arm of fifty human volunteers aged 18 to 65 years. The human volunteers were determined to be in good general health and free of any visible skin disease or anomaly in the area to be patched.

The patch was applied to a designated contact site and remained in place for 48 hours. At the end of this period, the patch was removed and the site was examined for any dermal response 1 hr, 24 hrs, and 48 hrs.

The dermal responses were scored according to the following grading scale:

Erythema Scale:

| | |
|---|---|
| 0 | Negative, normal skin |
| 1 | Definite erythema |
| 2 | Erythema and induration |
| 3 | Vesiculation |
| 4 | Bullous reaction |

The dermal responses of the 50 volunteers on all the sites were graded as 0 (no visible erythema or elevated response). None of the volunteers developed any irritation or skin sensitization sign. The results clearly indicated that the formulation did not sensitize the skin or produce allergic responses. Taken together, the results demonstrated that the formulation is safe for human use.

Additional safety studies were performed in humans using the Human Repeated Insult Patch Test also known as HRIPT or the Draize test (Draize et al., J. Pharm. Exp. Ther. 83: 377-390, 1944). The test challenged the development of Allergic Contact Dermatitis upon repeated applications of a test item under exaggerated exposure conditions.

In a study performed in compliance with Good Clinical Practice standards, the formulation of Example 1 was applied on the interscapular region of the back or on the arm of fifty human volunteers aged 18 to 65 years using the HRIPT. The human volunteers were determined to be in good general health and free of any visible skin disease or anomaly in the area to be patched.

During the Induction Phase, the patch was applied to a designated contact site and remained in place for 24 hours. At the end of this period, the patch was removed and the site was examined for any dermal response. The subject rested for 24 hours, after which the skin site was examined again. Another patch was then applied to the same site. The second application was identical to the first and remained in place for 24 hours. The procedure was repeated twice a week until a series of nine applications were made. The patch site was examined for any dermal response. The same site was used throughout the study.

During the Challenge Phase, after the 9th application, a rest period of 2 weeks elapsed after which a challenge application was applied in the same manner and to the same site described herein above and to a naïve site on the opposite site of the body. The challenge application was removed after 24 hours and the site was examined and graded for signs of irritation or sensitization. A follow-up examination was conducted at 24 hours after patch removal as well as at 48 hours and 72 hours.

The dermal responses were scored according to the following grading scale:

Erythema Scale:

| | |
|---|---|
| 0 | No visible erythema |
| 1 | Mild erythema (faint pink to definite pink) |
| 2 | Moderate erythema (definite redness) |
| 3 | Severe erythema (very intense redness) |

The results indicated that no signs of skin irritation were observed in any of the volunteers in the course of the nine applications of the Induction Phase. In addition, the formulation of Example 1 did not induce in the 10^{th} application (Challenge Phase) a contact dermal irritation and/or sensitization in human subjects. Thus, the formulation of Example 1 is safe for human use. As treatments with otic foams are expected to last a few days and may require repeated application of the medication on the same site (the inflamed ear), these results indicate that the formulation can be used for long periods of time without causing any irritation or any allergic responses. 20

### Example 4

### In Vitro Antibiotic Efficacy Studies

In order to demonstrate that the excipients used in the formulation of Example 1 do not interact with Ciprofloxacin or inhibit its antibiotic activity, an Antibiotics-Microbial Assay—Zone of Growth Inhibition was performed. In this assay the diameter (halo) of bacterial growth inhibition around filters soaked with a test item was measured and compared to a standard solution of antibiotic. For this end, sterile Microbiological cotton swabs were soaked with cultures of E. Coli (ATCC No. 8739), S. aureus (ATCC No. 6538) or of Pseudomonas aeruginosa (ATCC No. 9027) and evenly spread on top of LB-agar plates. Filters soaked with the tested formulation were placed on top of the plates and the plates were further incubated for 24 hrs. The diameter (halo) of growth inhibition around each filter was measured after 24 hrs of incubation at 37° C. A solution that contained the same concentration of Ciprofloxacin Hydrochloride (0.35%) as in the formulation tested was used as a control. The results are summarized in Table 2.

TABLE 2
Effect of different formulations on
the diameter of growth inhibition.

| | | Pseudomonas | |
|---|---|---|---|
| Sample | E. Coli | aeruginosa | Staph. Aureus |
| CDX24 | 3.45 cm (95.8%) | 4.7 cm (96.9%) | 5.0 cm (106.3%) |
| Ciprofloxacin | 3.6 cm (100%) | 4.85 cm (100%) | 4.7 cm (100%) |
| HCl solution | | | |
| (0.35%) | | | |

The results demonstrate that the antibiotic potency of the formulation tested was similar to the potency displayed by the commercially available ear drops (Table 2).

### Example 5

### Clinical Study

A clinical study for assessing the safety, efficacy, and clinical non-inferiority of once a day treatment with FoamOtic Cipro-Dexa (the formulation of Example 1 also designated CDX 24) in comparison to twice daily application of a commercial ciprofloxacin/dexamethasone ear drop suspension (Ciprodex of Alcon Labs) in the treatment of acute diffuse Otitis Externa (“Swimmer's ear”) was conducted. The study, open-label and randomized, enrolled 105 adult and children patients diagnosed with Otitis Externa of presumed bacterial origin. Inclusion and outcome criteria included the signs and symptoms of the disease: ear edema, ear pain, ear discharge, and tenderness to movement of the tragus/pinna. The study, complying with GCP guideline, was performed in 6 medical centers in Israel from July to October 2011. The patients were randomized into one of the two treatment groups: (1) Once a day treatment with FoamOtic Cipro-Dexa, i.e., the otic foam formulation designated CDX24 listed in Example 1 herein above (n=51) or (2) twice a day treatment with commercial ear drops (Ciprodex of Alcon Labs; n=54). Each medication contained ciprofloxacin and dexamethasone at the same concentration (ciprofloxacin 0.3% and dexamethasone 0.1%).

The patients applied the medications for 7 days. Upon completion of the treatment, the patients returned for a test-of-cure visit in which the status of the disease was assessed and compared to baseline. The patients were considered “Cured” if the Physician considered that no additional antibiotics treatment was needed. If all signs and symptoms of the disease were fully resolved, the patient was graded as Cured-Resolution) or if not all the symptoms were fully resolved but no additional antibiotics treatment was needed (Improvement). The patients were considered “Treatment Failure” if additional antibiotic therapy was needed.

As shown in Table 3 of the Intent-to-treat analysis, the percentage of cured patients was 92.2% in the foam treated group and 87% in the Ciprodex Group. The percentage of full Resolution cases with the foam applied once-daily was 84.4% versus 74.0% in the Ciprodex ear drops applied twice daily (Table 3). Treatment failure was 7.8% in the foam treated group and 13.0% in the Ciprodex group (Table 3).

TABLE 3
Summary of the clinical results - Intent-to-treat Analysis

| | FoamOtic Cipro-Dexa | Ciprodex Ear Drops |
|---|---|---|
| | Applied once-daily | Applied twice daily |
| Clinical Response | (n = 51) | (n = 54) |
| Cured (Resolution + | 92.2 | 87.0 |
| Improvement) | | |
| Failure | 7.8% | 13.0% |
| Resolution | 84.4% | 74.0% |

Thus, once-daily treatment with the Otic foam of the present invention was as efficient and even more efficient than twice daily application of commercial ear drops. Similar results were obtained by the per-protocol analysis (PP) which included 93 patients (FoamOtic Cipro Dexan=46 and Ciprodex n=47). As shown in Table 4, the 20 percentage of cured patients in the PP analysis was 97.8% in the once daily foam treated group and 93.6% in the twice daily Ciprodex Group. Treatment failure was 2.2% in the foam treated group and 6.4% in the Ciprodex group. Complete resolution of sings and symptoms of Acute Otitis Externa was obtained in 89.1% of the cases with the foam applied once-daily and 78.7% with the Ciprodex ear drops applied twice daily.

TABLE 4
Summary of the clinical results - Per protocol Analysis.

| | FoamOtic Cipro-Dexa | Ciprodex Ear Drops |
|---|---|---|
| | Applied once-daily | Applied twice daily |
| Clinical Response | (n = 46) | (n = 47) |
| Cured (Resolution + | 97.8 | 93.6 |
| Improvement) | | |
| Failure | 2.2% | 6.4% |
| Resolution | 89.1% | 78.7% |

In addition, there were no differences in the levels of cure between children and adults. Likewise, the microbial eradication levels were similar in both treatments.

Thus, the present results indicate the clinical superiority or non-inferiority of the otic foam formulation of the present invention as compared to commercial ear drops demonstrating that half the amount of ciprofloxacin provides the same relief when administered in the form of a foam as opposed to in the form of ear drops.

### Example 6

### Formulations of Other Quinolones and Steroidal Anti-Inflammatory Agents

Preparation of pharmaceutical compositions of other quinolones and anti-inflammatory agents is prepared according to the procedures disclosed herein above and particularly exemplified in Example 1:

### Ciprofloxacin—Hydrocortisone;

### Ciprofloxacin—Betamethasone Valerate;

Ciprofloxacin—Dexamethasone acetate;

### Ofloxacin—Hydrocortisone;

### Ofloxacin—Dexamethasone;

Ofloxacin—Betamethasone valerate;
Ofloxacin—triamcinolone acetonide;

### Ofloxacin—Hydrocortisone;

### Moxifloxacin—Dexamethasone;

### Moxifloxacin—Hydrocortisone;

Moxifloxacin—Betamethasone valerate;
Moxifloxacin—triamcinolone acetonide;

### Moxifloxacin—Hydrocortisone;

### Finafloxacin—Dexamethasone;

### Finafloxacin—Hydrocortisone;

Finafloxacin—Betamethasone valerate;
Finafloxacin—triamcinolone acetonide;

### Orbifloxacin—Mometasone Furoate;

### Orbifloxacin—Betamethasone Valerate;

### Orbifloxacin—Prednisolone Acetate;

Orbifloxacin—dexamethasone;

### Orbifloxacin—Hydrocortisone;

### Enrofloxacin—Mometasone Furoate;

### Enrofloxacin—Betamethasone Valerate;

### Enrofloxacin—Prednisolone Acetate;

### Enrofloxacin—Dexamethasone;

### Marbofloxacin—Dexamethasone;

### Marbofloxacin—Hydrocortisone;

Marbofloxacin—Flucinolone acetonide;

### Marbofloxacin—Prednisolone Acetate;

### Marbofloxacin—Mometasone Furoate;

### Marbofloxacin—Betamethasone Valerate;

### Marbofloxacin—Dexamethasone.

It will be appreciated by persons skilled in the art that the present invention is not limited by what has been particularly shown and described herein above, rather the scope of the invention is defined by the claims that follow.

1-32. (canceled)

33. A foamable pharmaceutical composition comprising:
(i) a polar co-solvent in an amount of from about 40% (w/w) to about 70% (w/w) of the composition;
(ii) water in an amount of from about 30% (w/w) to about 60% (w/w) of the composition;
(iii) a surfactant and/or a foaming agent;
(iv) a stabilizing agent;
(v) a quinolone;
(vi) a steroidal anti-inflammatory agent; and
(vii) a compressed propellant gas
wherein the composition is devoid of oil, and wherein the composition packaged in a container is adapted to form foam after being dispensed from the container.

34. The foamable pharmaceutical composition according to claim 33 wherein the polar co-solvent is selected from the group consisting of propylene glycol, polyethylene glycol, polypropylene alkyl ether, and combinations thereof.

35. The foamable pharmaceutical composition according to claim 33, wherein the polar co-solvent is propylene glycol.

36. The foamable pharmaceutical composition according to claim 33, wherein the polar co-solvent and water are present in a ratio of from about 2:1 to about 2:3.

37. The foamable pharmaceutical composition according to claim 33, wherein the surfactant is a synthetic surfactant selected from the group consisting of polysorbate 20, polysorbate 60, polysorbate 80, glyceryl stearate, polyoxyl 40 stearate, and combinations thereof.

38. The foamable pharmaceutical composition according to claim 33, wherein the surfactant is present in the composition in an amount ranging from about 2% (w/w) to about 4% (w/w) of the composition.

39. The foamable pharmaceutical composition according to claim 33, wherein the foaming agent is a long chain alcohol.

40. The foamable pharmaceutical composition according to claim 39, wherein the long chain alcohol is cetyl alcohol.

41. The foamable pharmaceutical composition according to claim 33, wherein the foaming agent is present in an amount ranging from about 0.1% (w/w) to about 0.5% (w/w) of the composition.

42. The foamable pharmaceutical composition according to claim 33, wherein the stabilizing agent is a polymeric agent selected from natural polymers, semi-synthetic polymers and synthetic polymers.

43. The foamable pharmaceutical composition according to claim 33, wherein the quinolone is selected from the group consisting of ciprofloxacin, ofloxacin, moxifloxacin, levofloxacin, lomefloxacin, nadifloxacin, norfloxacin, pefloxacin, rufloxacin, balofloxacin, gatifloxacin, grepafloxacin, levofloxacin, pazufloxacin, sparfloxacin, temafloxacin, tosufloxacin, clinafloxacin, gemifloxacin, sitafloxacin, trovafloxacin, prulifloxacin, garenoxacin, delafloxacin, marbofloxacin, enrofloxacin, danofloxacin, difloxacin, ibafloxacin, orbifloxacin, sarafloxacin and pharmaceutically acceptable salts thereof.

44. The foamable pharmaceutical composition according to claim 33, wherein the quinolone is ciprofloxacin hydrochloride.

45. The foamable pharmaceutical composition according to claim 33, wherein the steroidal anti-inflammatory agent is selected from the group consisting of dexamethasone, hydrocortisone, prednisolone, methylprednisolone, prednisone, triamcinolone, mometasone, budesonide, fluocinolone, betamethasone, cortisone isoflupredone, tixocortol, triamcinolone, amcinonide, desonide, fluocinonide, halcinonide, fluocortolone, aclometasone, clobetasone, fluocortolone, fluocortolone, and fluprednidene, and salts thereof.

46. The foamable pharmaceutical composition according to claim 33, wherein the steroidal anti-inflammatory agent is dexamethasone.

47. The foamable composition according to claim 33, comprising:
(i) propylene glycol in an amount of from about 40% (w/w) to about 70% (w/w) of the composition;
(ii) water in an amount of from about 30% (w/w) to about 60% (w/w) of the composition;
(iii) a surfactant and/or a foaming agent;
(iv) a stabilizing agent;
(v) a quinolone;
(vi) a steroidal anti-inflammatory agent; and
(vii) a compressed propellant gas
wherein the composition is devoid of oil, and wherein the composition packaged in a container is adapted to form foam after being dispensed from the container.

48. The foamable pharmaceutical composition according to claim 33, comprising:

| Ingredient | w/w (%) |
|---|---|
| Propylene glycol | 49.00 |
| Water | 46.38 |
| Polysorbate 80 | 2.50 |
| PEG 40 stearate | 0.80 |
| Cetyl alcohol | 0.40 |
| Hydroxyethyl cellulose | 0.20 |
| Glycerol | 0.10 |
| Ciprofloxacin HCl | 0.35 |
| Dexamethasone base | 0.10 |
| Sodium acetate | 0.15 |
| Acetic acid | q.s. |
| Benzalkonium chloride | 0.02 |

q.s. = quantity sufficient to obtain the desired pH.

49. A method for treating an ear disorder comprising administering to the ear of a subject in need of such treatment a therapeutically effective amount of a foamable pharmaceutical composition according to claim 33.

50. The method according to claim 49, wherein the subject is a human.

51. The method according to claim 49, wherein the subject is an animal.

52. The method according to claim 49, wherein the ear disorder is otitis selected from the group consisting of otitis externa and otitis media.
